(19) United States
(12) Patent Application Publication
(10) Pub. No.: US 2010/0114129 A1
(43) Pub. Date: May 6, 2010

(54) TISSUE JOINING DEVICES CAPABLE OF DELIVERY OF BIOACTIVE AGENTS AND METHODS OF USE THEREOF

(76) Inventors: Margaret A. Wheatley, Media, PA (US); Ari D. Brooks, Cherry Hill, NJ (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/307,976

(22) PCT Filed: Jul. 17, 2007
(86) PCT No.: PCT/US07/16211

§ 371 Date: Sep. 29, 2009

Related U.S. Application Data
(60) Provisional application No. 60/832,216, filed on Jul. 20, 2006.

(51) Int. Cl. A61B 17/11; A61K 9/00; A61K 38/16; A61K 38/02

(52) U.S. Cl. 606/153; 424/423; 514/12; 514/2

(57) ABSTRACT

The instant invention concerns a device for joining tissue comprising a ring and rivet, the ring comprises a biocompatible and biodegradable polymer and contains at least one bioactive agent. The rivet has a hollow lumen. The invention also relates to methods of making such devices and the use of such devices in joining tissue.

### CROSS-REFERENCE TO RELATED APPLICATIONS

This application claims the benefit of U.S. Provisional Application Ser. No. 60/832,216, filed Jul. 20, 2006, the disclosure of which is incorporated herein by reference in its entirety.

### FIELD OF THE INVENTION

The instant invention relates to biodegradable tissue joining devices which are capable of delivering bioactive agents to the surrounding tissue. The invention also relates to use of such devices.

### BACKGROUND OF THE INVENTION

A number of medical products using biodegradable polymers are known. These include sutures, clips and anchors, hemostatics, and adhesion barriers. Although absorbable staples have existed for over twenty years (Hirashima T, Eto T, DenBesten L. Lactomer, American Journal of Surgery 1985; 150 (3):381-385), currently, most surgeons use stainless steel or titanium staples because there has been no proven benefit to an absorbable staple.

Surgical staplers revolutionized abdominal surgery in the 1970s (Ravitch M M, Steichen F M, Adv Surg 1984; 17:241-279). They are the fastest and most reliable way to anastomose bowel and resect soft tissue and they are well suited to minimally invasive surgical approaches.

Historically, surgical procedures that require anastomosis of small lumen structures, such as the common bile duct, ureters, and blood vessels have not benefited from the reliability and ease of surgical stapling. Current barriers to the use of surgical staples in small structures include their size and susceptibility to ill effects from scarring at the tissue joint. When a structure is divided, its blood supply is divided as well making the end very tenuous. This end is then joined to another structure with reduced vascularity. With a thin wall, small lumen size and reduced blood flow the risk of poor healing, leak or late stricture is very high (Adzick N S. Wound Healing. In: Sabiston Jr D C, Lyerly H K, editors. Textbook of surgery: the biological basis of modern surgical practice. Philadelphia: W.B. Saunders Company, 1997: 207-220).

Thus, there is a need in the art for improved staples that are suitable for small structures.

### SUMMARY OF THE INVENTION

In some embodiments, the invention concerns a device for joining tissue comprising a rivet having first and second ends and a generally cylindrical portion connecting the ends, a lumen being in communication with each of the ends through the cylindrical portion, the first end of the rivet having a larger diameter than the cylindrical portion, the cylindrical portion having at least one corrugation external to the rivet; and a ring for cooperation with the corrugations of the rivet such than when urged over the corrugations, the ring remains affixed to the rivet; the ring comprising a first biodegradable polymer having bioactive composition releasably contained therein.

In some preferred embodiments, the surface of the rivet and/or ring contain one or more bonding entities on its surface. In some embodiments, the bonding entity is polyethylene glycol or a cell-surface receptor recognition sequence. In certain embodiments, the polyethylene glycol is grafted to the surface of the ring or rivet. In some embodiments of the invention, the cell-surface receptor recognition sequence is Arg-Gly-Asp.

In certain embodiments, the rivet has at least one suturable location on the larger diameter portion of the rivet.

In some preferred embodiments, all portions of the device are sterilizable.

Some preferred embodiments utilize a first polymer that is polyester, polylactide, polyglycolide, ε-polycaprolactone, copolymer of polylactide and polyglycolide, copolymer of lactide and lactone, polysaccharide, polyanhydride, polystyrene, polyalkylcyanoacrylate, polyamide, polyphosphazene, poly(methylmethacrylate), polyurethane, copolymer of methacrylic acid and acrylic acid, copolymer of hydroxyethylmethacrylate and methylmethacrylate, polyaminoacid, polypeptide, natural or synthetic polysaccharides, or combinations of blends thereof. In some of these embodiments, the first polymer is polylactic co-glycolic acid (PLGA).

In certain embodiments, the ring is made of an excipient and up to 50% by weight of a bioactive agent. In some of these embodiments, the ring contains less than 50% by weight, or less than 25% by weight, or less than 10% by weight, or less than 5% by weight of biodegradable polymer. In some embodiments, the ring is substantially free of such polymers. By substantially free, it is intended to mean less than 1% by weight based on the weight of the ring.

The rivet can comprise a biodegradable polymer. This polymer can comprise the polymers discussed for the polymer used for the ring and can be the same or different than the polymer used for the ring.

Based on the end uses of these devices, it is preferred that the polymers used with respect to this invention are FDA-approved biocompatible, biodegradable polymer.

The rivet can also comprise one or more bioactive agents. The bioactive agent of the ring and rivet can be one or more pharmaceutical compounds and can be the same or different. These compounds include antibiotics, anti-inflammatory agents, anti-cancer agents, growth factors, proteins, peptides, tyrosine kinase inhibitors and other molecular directed therapeutics.

In some embodiments, the rivet has an exterior wall and said exterior wall has one or more ridges.

The invention also concerns methods of joining tissues using the devices described herein. In one embodiment, the tissue comprises a tubular structure, and the method comprises:

securing a rivet into the tubular tissue portion, the rivet comprising first and second ends and a generally cylindrical portion having at least one corrugation, a lumen being in communication with each of the ends through the cylindrical portion; the securing being of the first end of the rivet, said first end having a larger diameter than the cylindrical portion;

placing a ring sized for cooperation with the corrugation adjacent to the second portion of tissue; and

urging the ring and second portion of tissue against the second end of the rivet such that the ring becomes entrapped on the rivet by the corrugation.

The invention also concerns a method for making a component for interaction with a biocompatible rivet. In one embodiment, the method comprises:

blending biodegradable polymer with up to about 50 percent by weight of the blend of a bioactive agent compatible with the polymer; and

compressing the blend to form a toroidal or flat cylindrical shaped body having a preselected degree of compression.

In some embodiments, the method further comprises comminuting the blend or components of the blend and providing the materials of the blend in a particle size range, measured by sieving, of between 60 and about 250 microns. The compression can also provide the shaped body in a form adapted for snappably interacting with the biocompatible rivet. In some preferred embodiments, the shaped body is capable of reversibly deforming in an outward, axial direction in order to snap over a corrugation in the rivet.

In some embodiments, the cylindrical portion of the rivet is non-circular. In such a device, the cylindrical or elongated portion of the rivet can be in the shape of a square, rectangle, hexagon, or other geometric shape that is useful for the staple. The ring is shaped to accommodate the shape of the rivet.

### BRIEF DESCRIPTION OF THE DRAWINGS

FIG. 1 illustrates one embodiment of a ring and button (or rivet).

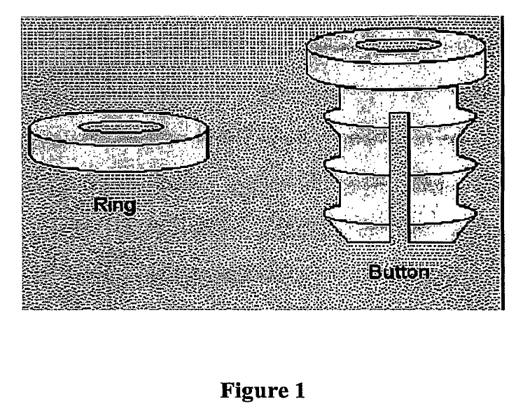

FIG. 2 shows an illustrative die and upper and lower presses. An illustration of a ring is also provided.

### DETAILED DESCRIPTION OF ILLUSTRATIVE EMBODIMENTS

The present invention allows biodegradable staples to achieve their true potential by using bio-absorbable polymer to serve as a drug delivery vehicle.

Use at small, delicate sites is one area that an absorbable staple can truly revolutionize surgical interventions. Liver transplantation, surgical resection for hepatocellular cancer, pancreatic cancer, and cholangio-carcinoma are just a few of the procedures that may require anastomosis of the common bile duct. The standard protocol is to hand-sew the common bile duct either to itself or its enteric counterpart. Due to the narrow lumen of the common bile duct, hand-sewn biliary anastomosis is technically challenging, extremely time-consuming, and may lead to lifethreatening complications if it fails (Yeo C J, Cameron J L, Sohn T A, Lillemoe K D, Pitt H A, Talamini M A et al., Ann Surg 1997; 226 (3):248-257).

There is a great deal of in vivo data demonstrating improvements in anastomotic burst strength using a variety of wound modulating substances ranging from transforming growth factor β (Ciacci C, Lind S E, Podolsky D K, Gastroenterology 1993; 105 (1):93-101; Shah M, Revis D, Herrick S, Baillie R, Thorgeirson S, Ferguson M et al., Am J Pathol 1999; 154 (4):1115-1124; Slavin J, Nash J R, Kingsnorth A N., British Journal of Surgery 1992; 79 (1):69-72), to insulin-like growth factor (Chen K, Nezuu R, Wasa M, Sando K, Kamata S, Takagi Y et al., Journal of Parenteral & Enteral Nutrition 1999; 23 (5:Suppl):Suppl-92) and even antibodies that block cytokines which inhibit wound healing (Shah M, Foreman D M, Ferguson M W, J Cell Sci 1994; 107 (Pt 5):1137-1157). Although there are many topical wound products for use on the skin based on these positive effects (Ehrlich H P, Freedman B M, Cytokines Cell Mol Ther 2002; 7 (3):85-90; Landi F, Aloe L, Russo A, Cesari M, Onder G, Bonini S et al., Ann Intern Med 2003; 139 (8):635-641), these applications were never translated into a clinical product for anastomoses because of the difficulties involved in delivering these agents to the site of interest.

### Biodegradable Polymers

The instant invention provides a prototype new staple for small lumen structures fabricated from an FDA-approved biocompatible, biodegradable polymer that delivers bioactive agents at a pre-programmed rate to improve healing for in vivo proof of principle. This new staple is composed of two parts; a rivet with a hollow lumen for flow of bodily fluids and a ring to help secure the approximated tissues in place. The biocompatible staple will degrade, simultaneously delivering drugs such as wound healing modulators (such as TGFβ and keratinocyte growth factor (KGF)) that would prevent scarring or tissue breakdown and their attendant complications until the necessary healing has taken place and continuity has been restored. Other drugs may be used to provide other benefits to the patient.

Biodegradable polymer may be selected from, but not limited to, polylactides, polyglycolides, polycaprolactones, copolymers of polylactide and polyglycolide, copolymers of lactide and lactone, polysaccharides, polyanhydrides, polystyrenes, polyalkylcyanoacrylates, polyamides, polyphosphazenes, poly(methylmethacrylate)s, polyurethanes, copolymers of methacrylic acid and acrylic acid, copolymers of hydroxyethylmethacrylate and methylmethacrylate, polyaminoacids, polypeptides, and natural and synthetic polysaccharides, or combinations and blends thereof. In some embodiments, preferred polymers are those which are biocompatible and biodegradable. In some preferred embodiments, the polymers are FDA approved. In one preferred embodiment the polymer is polylactic co-glycolic acid (PLGA).

Methods for preparing PLGA are discussed in Davis, et al, J. International Journal of Pharmaceutics 2002, 248, 149-56. It should be noted the properties of the polymers can be altered by the ratio of monomers and by the molecular weight. For example, changing the ratio of monomer units in the backbone of the polymer will create different drug release profiles. In some compositions, lactic acid is used as the predominant monomer due to its hydrophobic nature.

### Bioactive Agents

Any drug or pharmaceutical product that can benefit the patient can be used with the devices of the invention. Suitable pharmaceuticals include, but not limited to classes of antibiotics, anti-inflammatory agents, anti-cancer agents, growth factors, proteins, and peptides.

Examples of bioactive agents which can be loaded into the devices include, but are not limited to: antineoplastic and anticancer agents such as azacitidine, cytarabine, fluorouracil, mercaptopurine, methotrexate, thioguanine, bleomycin peptide antibiotics, podophyllin alkaloids such as etoposide, VP-16, teniposide, and VM-26, plant alkaloids such as vincristine, vinblastine and paclitaxel, alkylating agents such as busulfan, cyclophosphamide, mechlorethamine, melphalan, and thiotepa, antibiotics such as dactinomycin, daunorubicin, plicamycin and mitomycin, cisplatin and nitrosoureases such as BCNU, CCNU and methyl-CCNU, anti-VEGF molecules, gene therapy vectors and peptide inhibitors such as MMP-2 and MMP-9, which when localized to tumors prevent tumor growth; inflammatory modulators such as cytokines in the TGF-beta family and cyclooxygenase inhibitors; antibiotics and anti-fungals such as beta-lactams, macrolides, lincosamides, aminoglycosides, tetracyclines, polypeptides, sulfonamides, and fluoroquinolines; wound healing agents such as TGF-β, PDGF, EGF, TGF-α, VEGF, IGF-1, FGFs, angiopoietin, KGF, endothelin, TNF-α, Interleukin-1 or -1β, Interleukin-4, Interleukin-6, Interleukin-8, Interleukin-10, Interleukin-18, SLPi, MCP-1, MIP-1α, MIP-2, IFN-α, IFN-β, and IFN-γ; adhesion molecules such as VCAM-1, ICAM-1, ELAM-1, integrins, selectins, and immunoglobulin superfamily; nucleic acids such as mRNA, DNA, antisense oligonucleotides, plasmids and vectors; vaccines such as DNA and RNA vaccines, peptides, immunostimulatory molecules, and modified bacterial and viral agents; immune regulators such as antibodies, glucocortocoids, immunosuppressants, and anti-idiotype antibodies; endocrine agents such as insulin, thyroid hormone, steroid hormones, androgens, estrogens, and somatostatin; coagulation modulators such as heparin, fractionated or unfractionated, anti-platelet agents, thrombolytic agents, streptokinase, urokinase, and tissue plasminogen activator; vascular tone modulators such as nitric oxide, N-omega-nitro-L-arginine methyl ester, alpha or beta agonists, thrombin and fibrin; and proteoglycans and glycosaminoglycans including hyaluronic acid. Additional agents include tyrosine kinase inhibitors and other molecular directed therapeutics.

By “bioactive agent” it is also meant to be inclusive of imaging or labeling agents for post-insertion visualization of the tissue joining device or surrounding area. For example, radiopaque markers for visualization by X-ray may be loaded into one or more of the interconnecting components of the closing means. Gas bubbles can also be loaded into one or more of the connecting components for visualization by ultrasound. Radionuclides can be loaded into one or more of the components for visualization using nuclear medicine such as gamma emitters such as ⁹⁹Tc, or ¹²⁵I. In addition, a fluorophore can be loaded into one or more of the connecting components for visualization via fluorescence detection. Further beta emitters such as ¹⁸F as in ¹⁸F-FDG can be loaded for PET scans.

### Drug-Eluting Rings

As used herein, the term ring encompasses joining devices where the external shape of the ring need not be round. This shape may be a square, rectangle, hexagon, or the like.

The polymer can be ground (pestle and mortar) or milled or dissolved and lyophylised or rendered divided into fragments by standard methods familiar to those experienced in the art. In some embodiments, the polymer can be sieved prior to use. In certain embodiments, the sieve is 60-250 microns. In one embodiment, the polymer is sieved through a 125 micron mesh. Other sieving sizes may be used depending on the needs of the device.

In some embodiments the drug or pharmaceutical agent is ground (pestle and mortar, for example) or milled or rendered divided into fragments by standard methods familiar to those experienced in the art. In some preferred embodiments, the agents are sieved. In certain embodiments, the sieve is 60-250 microns. One preferred embodiment uses 125 micron mesh sieving. Other sieving sizes may be used depending on the needs of the device. It is also contemplated that the polymer and the bioactive agent can be comminuted as the blend and, optionally, sieved.

Drug and polymer and intimately mixed in ratios preferred but not limited to between 0% to 80% by weight of drug. In some preferred embodiments the amount of drug is from about 1 to about 50% by weight. In yet other embodiments, the amount of drug is from about 1 to about 25% by weight. In still other embodiments, the amount of drug is from about 1 to about 10% by weight. The release profile of the drug can be altered by adjusting the loading of the drug (ie, the amount of drug) in the device.

The ring can be made by blending biodegradable polymer with up to about 50 percent by weight of the blend of a bioactive agent compatible with the polymer; and compressing the blend to form a toroidal or flat cylindrical shaped body having a preselected degree of compression. The method can further comprise comminuting the blend or components of the blend and providing the materials of the blend in a particle size range, measured by sieving, of between 60 and about 250 microns. The compression provides the shaped body in a form adapted for snappably interacting with the biocompatible rivet which is capable of reversibly deforming in an outward, axial direction in order to snap over a corrugation in the rivet.

The mixture of polymer and bioactive material can be weighed and loaded into a custom designed die-and-punch tool to an amount that will results in a ring of the desired dimensions. In some embodiments, the ring preferably has a 10 mm or less outer diameter, 2 mm or less height, and a 7 mm or less center hole diameter. In other embodiments, such as for bowel anastamosis, the ring will be preferably larger.

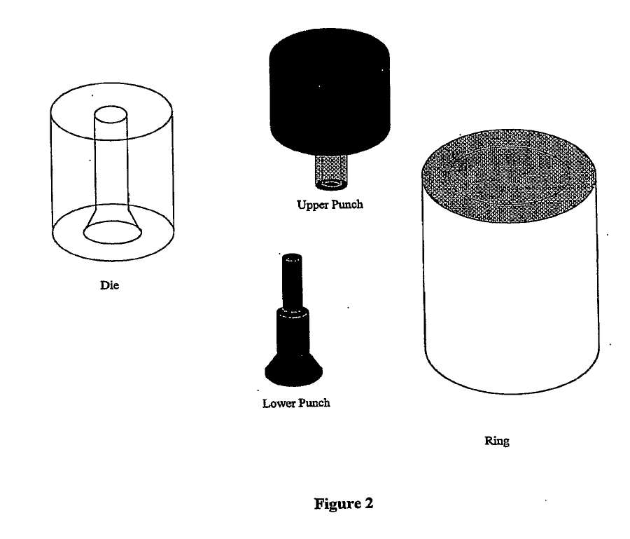

The die-and-punch can then be placed in a compression device (e.g. Carver hydraulic press) and compressed to the desired pressure (preferably 0.1 to 1 metric ton) for the desired time (preferably 10 s to one minute). The compressed ring, of the desired size and compaction can then be released from the die- and -punch for use. One such die-and-punch set is illustrated in FIG. 2.

In an alternate embodiment, the die-and-punch can be loaded with microcapsules composed of polymer and drug manufactured by any standard microencapsulation technique familiar to one skilled in the art.

In some embodiments, the loaded die and punch can be heated during compression.

In yet another embodiment, a sheet of compressed polymer and drug can be formed by methods known to those skilled in the art, and a ring can be stamped out with a custom-built stamp. It is preferred that the sheet be flat—being uniform in thickness.

In still other embodiments, the ring can be made by a method similar to that taught by Choonara, et al, International Journal of Pharmaceutics 2006, 310, 15-24 which teaches production of doughnut shaped minitablets.

In some embodiments, an additional drug-loaded ring or disc can be inserted between button and rivet. The composition of the additional ring (polymer and drug) includes those described herein for the ring.

In some embodiments, the average ring contains about 0.1 g polymer. With a drug loading of 10% by weight, this represents about 10 mg drug. If the ring loses about 3% of its weight per day, the drug dose is about 0.3 mg/day. Depending on the dosage desired, the amount of drug loaded can be altered or the composition of the ring can be varied to increase or decrease the drug release.

### The Rivet

The rivet is sometimes referred to as the button. The terms are used interchangeably herein. The rivet is preferred to have a hollow lumen to allow fluid to flow through the rivet. In some preferred embodiments, the rivet has one or more ridges or corrugations on the exterior wall to assist is securing the ring when the rivet is inserted into the opening of the ring. The rivet can be made by standard procedures known to those skilled in the art. In some preferred embodiments, the rivet is made of a biodegradable polymer. Suitable polymers include those discussed herein in regard to the ring.

In some preferred embodiments, the rivet utilizes the same polymer as the ring.

In certain embodiments, one or more bioactive agents can also be included in the rivet. These agents include those discussed herein for the ring and can be incorporated in amounts described in regard to the ring. If an agent is included in the ring and rivet, the agent can be the same or different. Mixtures of agents, rather than a single drug, can also be included in either the ring or rivet.

In some embodiments, the rivet has corrugations that extend around the elongated portion of the rivet but do not extend in a continuous manner from the second end toward the first end. In yet other embodiments, the rivet and ring can interact by a screw/nut mechanism. In such an embodiment, the rivet corresponds to an externally spirally grooved hollow cylinder having a first and second end as described herein and the corrugations can run from the second end toward the first end of the elongated portion of the rivet. In this latter embodiment, the ring has an internal screw thread that is compatible with the grove of the rivet.

In yet other embodiments, one or more pins project from the under surface of the upper flange (first end of the rivet having a larger diameter) of the rivet and can be used to help secure the tissue. In certain embodiments, these pins can fit into holes aligned in the ring portion.

### Bonding Entities

The rings and/or rivets described herein can have one or more bonding entities on their surface. Bonding entities are compounds or moieties that either prevent fouling of the surface or can assist in promoting healing. Such entities can be absorbed onto the surface or chemically bonded to the surface. In some embodiments, the entity is polyethylene glycol (PEG) or a cell-surface receptor recognition sequence. PEGs of various molecular weights are believed to be useful to prevent fouling of the device surface. PEGs have been used in controlled drug delivery methods. See, for example, Mosqueira, et al., Biomaterials 2001, 22, 2967-2979. Cell-surface receptor recognition sequences, such as RGD peptide (Arg-Gly-Asp) are believed to promote cell adhesion. See for example, Massia, et al., Analytical Biochemistry 1990, 187, 292-301 and Hynes, Nature Medicine 2002, 8, 918-921.

### Methods of Utilizing the Ring and Rivet Tissue Joining Device

One illustrative method of using the ring and rivet concerns inserting the first end of the rivet into the tissue (such as into the bile duct) and securing thereto. The second end of the rivet is then inserted into the second tissue (the bowel, for example). The ring can then be pushed onto the rivet. The ring is typically positioned above one or more groves or corrugations on the rivet to secure the second tissue to the first tissue by using the ring to assist in trapping the tissue.

In some embodiments, the rivet and ring can be utilized with a stapler device. These staplers include, but are not limited to, circular anastamotic staplers, linear cutting staplers and linear apposition staplers. All three configurations are used for anastomosis, the last two are used for tissue transaction and sealing. Such procedures are known to those skilled in the art.

### Illustrative Uses and Utility of the Rivet and Ring Joining Devices

Potential end uses include surgery in bile duct, ureter, blood vessels, or any site where conventional staplers are used. Another potential end uses include laparoscopy and natural orifice endoscopic surgery. Such uses may contribute to improved morbidity/outcomes. Among the drugs that can be delivered are wound healing modulators, antibiotics, chemotherapeutic agents, radioactive, fluorescent, or contrast agents for imaging, vaccines, immunotherapy, or gene therapy.

In some embodiments, the devices described herein are used in conjunction with robotics. Surgical techniques using robotics are well known to those skilled in the art.

Use of the joining devices described herein are believed to be able to shorten operative time and accuracy in small structures, lower the rate of recurrent tumors, lower the rate of infection or leak in an anastomosis, and improve healing and outcomes of surgery overall.

All patents and publications disclosed herein are incorporated by reference in their entirety.

What is claimed:

1. A device for joining tissue comprising:
a rivet having first and second ends and a generally cylindrical portion connecting the ends, a lumen being in communication with each of the ends through the cylindrical portion, the first end of the rivet having a larger diameter than the cylindrical portion, the cylindrical portion having at least one corrugation external to the rivet; and
a ring for cooperation with the corrugations of the rivet such than when urged over the corrugations, the ring remains affixed to the rivet;
the ring comprising a first biodegradable polymer having bioactive composition releasably contained therein.

2. The device of claim 1 having at least one suturable locations on the larger diameter portion of the rivet.

3. the device of claim 1, all portions of which are sterilizable.

4. The device of claim 1 wherein said first biodegradable polymer is polyester, polylactide, polyglycolide, ε-polycaprolactone, copolymer of polylactide and polyglycolide, copolymer of lactide and lactone, polysaccharide, polyanhydride, polystyrene, polyalkylcyanoacrylate, polyamide, polyphosphazene, poly(methylmethacrylate), polyurethane, copolymer of methacrylic acid and acrylic acid, copolymer of hydroxyethylmethacrylate and methylmethacrylate, polyaminoacid, polypeptide, natural or synthetic polysaccharides, or combinations of blends thereof.

5. The device of claim 1 wherein said first biodegradable polymer is polylactic co-glycolic acid (PLGA).

6. The device of claim 1 wherein said bioactive agent is a pharmaceutical.

7. The device of claim 6 where said pharmaceutical is one or more of antibiotics, anti-inflammatory agents, anti-cancer agents, growth factors, proteins, and peptides.

8. The device of claim 6 where said pharmaceutical comprises growth factors.

9. The device of claim 1 where the rivet comprises a second polymer that is biocompatible and biodegradable.

10. The device of claim 9 where said second polymer is polyester, polylactide, polyglycolide, ε-polycaprolactone, copolymer of polylactide and polyglycolide, copolymer of lactide and lactone, polysaccharide, polyanhydride, polystyrene, polyalkylcyanoacrylate, polyamide, polyphosphazene, poly(methylmethacrylate), polyurethane, copolymer of methacrylic acid and acrylic acid, copolymer of hydroxyethylmethacrylate and methylmethacrylate, polyaminoacid, polypeptide, natural or synthetic polysaccharides, or combinations of blends thereof.

11. The device of claim 9 where said second polymer is polylactic co-glycolic acid (PLGA).

12. The device of claim 9 wherein said rivet comprises a bioactive agent.

13. The device of claim 12 wherein said bioactive agent comprises a pharmaceutical.

14. The device of claim 13 where said pharmaceutical is one or more of antibiotics, anti-inflammatory agents, anti-cancer agents, growth factors, proteins, and peptides.

15. The device of claim 1 further comprising at least one of the ring and rivet having one or more bonding entities on its surface.

16. The device of claim 15 where the bonding entity is polyethylene glycol or a cell-surface receptor recognition sequence.

17. The device of claim 16 where the polyethylene glycol is grafted to the surface of the ring or rivet.

18. The device of claim 17 where the cell-surface receptor recognition sequence is Arg-Gly-Asp.

19. The device of claim 1 further comprising a second ring capable of being positioned between the first end of the rivet and the ring, the second ring comprising a biodegradable polymer and a bioactive agent.

20. The device of claim 1 wherein at least one of the ring and rivet are coated with a bioactive agent.

21. A device for joining tissue comprising:
a rivet having first and second ends and a generally cylindrical portion connecting the ends, a lumen being in communication with each of the ends through the cylindrical portion, the first end of the rivet having a larger diameter than the cylindrical portion, the cylindrical portion having at least one corrugation external to the rivet; and
a ring for cooperation with the corrugations of the rivet such than when urged over the corrugations, the ring remains affixed to the rivet;
the ring comprising an excipients having bioactive composition releasably contained therein.

22. The device of claim 21 where the bioactive agent is one or more of antibiotics, anti-inflammatory agents, anti-cancer agents, growth factors, proteins and peptides

23. A method of joining two portions of tissue, at least one portion being tubular comprising: securing a rivet into the tubular tissue portion, the rivet comprising first and second ends and a generally cylindrical portion having at least one corrugation, a lumen being in communication with each of the ends through the cylindrical portion; the securing being of the first end of the rivet, said first end having a larger diameter than the cylindrical portion;
placing a ring sized for cooperation with the corrugation adjacent to the second portion of tissue; and
urging the ring and second portion of tissue against the second end of the rivet such that the ring becomes entrapped on the rivet by the corrugation.

24. The method of claim 23 wherein the joining of tissue is performed in a laparoscopy.

25. The method of claim 23 wherein the joining of tissue is preformed using robotics.

26. A method for making a component for interaction with a biocompatible rivet comprising:
blending biodegradable polymer with up to about 50 percent by weight of the blend of a bioactive agent compatible with the polymer; and
compressing the blend to form a toroidal or flat cylindrical shaped body having a preselected degree of compression.

27. The method of claim 26 further comprising comminuting the blend or components of the blend and providing the materials of the blend in a particle size range, measured by sieving, of between 60 and about 250 microns.

28. The method of claim 26 wherein the compression provides the shaped body in a form adapted for snappably interacting with the biocompatible rivet.

29. The method of claim 26 wherein the shaped body is capable of reversibly deforming in an outward, axial direction in order to snap over a corrugation in the rivet.

30. The device of claim 1 wherein the cylindrical portion of the rivet is non-circular.

31. The device of claim 1 wherein the corrugations of the rivet comprise an external spiral groove extending from the second end toward the first end and the ring comprises an internal screw thread that is compatible with the grove of the rivet.
